(19) United States
(12) Patent Application Publication
(10) Pub. No.: US 2008/0300210 A1
(43) Pub. Date: Dec. 4, 2008

(54) Method of Controlling Insects and Virus Transmission

(75) Inventors: Thomas L. German, Hollandale, WI (US); Anna E. Whitfield, Manhattan, KS (US)

Correspondence Address: QUARLES & BRADY LLP, 411 E. WISCONSIN AVENUE, SUITE 2040, MILWAUKEE, WI, 53202-4497, US

(73) Assignee: WISCONSIN ALUMNI RESEARCH FOUNDATION, Madison, WI (US)

(21) Appl. No.: 12/121,400

(22) Filed: May 15, 2008

Related U.S. Application Data
(60) Provisional application No. 60/938,345, filed on May 16, 2007.

(51) Int. Cl. A61K 31/7088; C12N 15/31; C12N 15/33; A01P 1/00; A01P 7/04; A01H 5/00

(52) U.S. Cl. 514/44; 536/23.71; 536/23.72; 800/302; 800/301; 800/279

(57) ABSTRACT

The present invention provides methods and compositions for controlling insects and virus transmission, including a genetic construct for inhibiting virus transmission by an arthropod, a transgenic plant, plant cell or plant tissue, a method of preventing transmission of an arthropod-dependent viral plant disease, a method of delivering an active toxic fragment of a Bacillus thuringinsis (Bt) toxin to an arthropod, and a biopesticidal composition for preventing transmission of an arthropod-dependent viral plant disease.

### CROSS-REFERENCE TO RELATED APPLICATIONS

This application claims priority to U.S. Provisional Application No. 60/938,345, filed on May 16, 2007, the entirety of which is hereby incorporated by reference herein for all purposes.

### STATEMENT REGARDING FEDERALLY SPONSORED RESEARCH OR DEVELOPMENT

This invention was made with United States government support awarded by the following agency: USDA AGRICCREE Grant Nos. 99-35303-8271 and 91-373002-6295. The United States government has certain rights in this invention.

### BACKGROUND

The invention relates generally to methods and compositions for controlling insect pests and plant viruses transmitted by such insects, and more particularly, for controlling insects of the order Thysanoptera (commonly called “thrips”) and plant viruses associated therewith.

Thrips, an arthropod, feed on a large variety of plants, principally feeding on stems, leaves, fruits and vegetables. Thrips particularly enjoy feeding on plants with commercial value such as peanuts, tomatoes, potatoes, eggplants and lettuce. Species of thrips include but are not limited to Frankliniella occidentalis, F. schultzei, F. fusca, F. tenuicornis, Thrips tabaci, T. setosus, T. moultoni, T. simplex, Scirtothrips dorsalis, Taeniothrips inconsequens and Anaphothrips obscurus. In addition, thrips spread plant diseases caused by over twenty plant-infecting viruses, including the Tomato Spotted Wilt Virus (TSWV) and the Impatiens Necrotic Spot Virus. See Jones D, “Plant viruses transmitted by thrips,” Eu. J. Plant Pathol. 113:119-157 (2005).

TSWV is a prominent plant pathogen with worldwide distribution. It infects at least 732 species of plants, causing monetary losses due to crop damage and pesticide application. TSWV is transmitted by thrips in a persistent, replicative manner due to the vector-virus relationship between thrips and TSWV. Although TSWV is acquired only during the larval stage, its transmission is due almost exclusively to adult thrips.

TSWV's life-cycle depends upon the following four steps. First, a larval thrips ingests TSWV from an infected plant. Second, TSWV then passes through the midgut wall of the larval thrips, where it replicates and spreads to surrounding muscle cells. Third, TSWV eventually makes its way into the salivary glands of the thrips as it develops. Finally, TSWF is delivered in a viable form into another host plant via salivary secretions by an adult thrips. Interestingly, adult thrips that acquire TSWV cannot transmit it.

Currently, thrips are controlled with chemical means (e.g., Orthene, Avid, Mesurol 75WP and Conserve SC), non-chemical/physical means (e.g., screens and proper sanitation) and biological means (e.g., predatory mites, predatory pirate bugs and soil-dwelling mites). Thrips, however, are tolerant of several insecticides and successful treatment often depends upon both the thrips species and the plant species, each of which may have a different treatment threshold.

Thrips and other arthropod vectors affect not only plants, but also animals, including mammals. While thrips and other arthropod vectors transmit more than 70% of viruses infecting plants, they also transmit more than 40% of viruses infecting mammals.

For the foregoing reasons, there is an on-going need for methods and compositions for controlling certain arthropods like thrips, as well as the viruses they transmit.

### SUMMARY OF THE INVENTION

In one embodiment, the present invention provides a genetic construct for inhibiting virus transmission by an arthropod comprising a first polynucleotide that encodes a midgut receptor binding domain of an envelope/membrane glycoprotein from a virus capable of infecting the arthropod and a second polynucleotide that encodes an active toxic fragment of a Bacillus thuringinsis (Bt) toxin. The genetic construct can further comprise a promoter operably linked to the first and second polynucleotides for expressing a fusion protein. In some embodiments, the arthropod is a member of Thysanoptera, such as thrips. In some embodiments, the virus is dependent upon a member of Thysanoptera for transmission to a host plant, such as the tomato spotted wilt virus (TSWV).

The nucleotide sequence for the envelope/membrane glycoprotein is selected from nucleotide sequences that encode amino acid sequences for G_{N} (SEQ ID NO:2), G_{C} (SEQ ID NO:3) or a functional fragment of the foregoing. In some embodiments, the nucleotide sequence for the envelope/membrane glycoprotein is selected from nucleotide sequences that encode an amino acid sequence for G_{N-S} (SEQ ID NO:4), G_{C-S} or a functional fragment of the foregoing.

In an alternate embodiment, the invention provides a transgenic plant, plant cell or plant tissue comprising a nucleic acid sequence encoding a soluble, envelope/membrane glycoprotein from a virus capable of infecting an arthropod operably linked to a plant-expressing promoter. In some embodiments, the arthropod is a member of Thysanoptera, such as thrips. In some embodiments, the virus is dependent upon a member of Thysanoptera for transmission to a host plant, such as the tomato spotted wilt virus. In some embodiments, the tissue is a seed. The nucleic acid soluble envelope/membrane glycoprotein is G_{N} (SEQ ID NO:2) and G_{C} (SEQ ID NO:3) or a fragment of the foregoing. The nucleic acid sequence encodes G_{N-S} (SEQ ID NO:4) G_{C-S} or a functional fragment of the foregoing. In some embodiments, the nucleic acid sequences further comprising an active toxic fragment of a Bt toxin.

In an alternate embodiment, the present invention provides a method of preventing transmission of an arthropod-dependent viral plant disease comprising the step of exposing an arthropod to a polypeptide that contains a midgut receptor binding domain of an envelope/membrane glycoprotein from a virus capable of infecting the arthropod in an amount sufficient to reduce viral binding.

In some embodiments, the polypeptide is provided in a solution or suspension and applied to plants or plant parts that the arthropod feeds upon.

In some embodiments, the polypeptide is provided in a transgenic plant.

In some embodiments, the soluble, envelope/membrane glycoprotein is provided in a transgenic plant.

In some embodiments, wherein the soluble, envelope/membrane glycoprotein is G_{N} (SEQ ID NO:2), G_{C} (SEQ ID NO:3) or a functional fragment of the foregoing.

In some embodiments, the soluble, envelope/membrane glycoprotein is G_{N-S} (SEQ ID NO:4), G_{C-S} or a functional fragment of the foregoing.

In some embodiments, the soluble, envelope/membrane glycoprotein further comprises an active toxic fragment of a Bt toxin.

In an alternate embodiment, the present invention provides a method of delivering an active toxic fragment of a Bacillus thuringinsis (Bt) toxin to an arthropod comprising the step of providing a polynucleotide that encodes a midgut receptor binding domain of an envelope/membrane glycoprotein from a virus capable of infecting the arthropod and a second polynucleotide that encodes an active toxic fragment of a Bacillus thuringinsis (Bt) toxin. In some embodiments, the arthropod is a member of Thysanoptera, such as thrips. In some embodiments, the virus is dependent upon a member of Thysanoptera for transmission to a host plant, such as the tomato spotted wilt virus.

In an alternate embodiment, the present invention provides a biopesticidal composition for preventing transmission of an arthropod-dependent viral plant disease comprising a genetic construct comprising a first polynucleotide that encodes a midgut receptor binding domain of an envelope/membrane glycoprotein from a virus capable of infecting the arthropod and a second polynucleotide that encodes an active toxic fragment of a Bacillus thuringinsis (Bt) toxin.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### DESCRIPTION OF PREFERRED EMBODIMENTS

The present invention provides methods and compositions for controlling insects and virus transmission.

### I. In General

In the specification and in the claims, the terms “including” and “comprising” are open-ended terms and should be interpreted to mean “including, but not limited to . . . . ” These terms encompass the more restrictive terms “consisting essentially of” and “consisting of.”

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. The Invention

The present invention provides methods and compositions for controlling insects and virus transmission.

### Genetic Construct.

In one embodiment the invention provides a genetic construct that includes a first polynucleotide that encodes a midgut receptor binding domain (or a functional fragment thereof) of an envelope/membrane glycoprotein from a virus capable of infecting the arthropod and a second polynucleotide that encodes an active toxic fragment of a Bacillus thuringinsis (Bt) toxin. By “active toxic fragment,” we mean that the fragment retains an ability to function as an insecticide against the arthropod. Such a construct is useful for making a fusion protein that can kill the arthropod and inhibit virus transmission by the arthropod. The genetic construct can optionally include a promoter operably-linked to the first and second polynucleotides for expressing a fusion protein. Cells that contain the above the genetic construct are within the scope of the present invention. Bt toxins and their functional domains are well known in the art.

The DNA and amino acid sequences of envelope/membrane glycoproteins as well as functional domains thereof of various viruses are known in the art. For example, the amino acid sequence of TSWV polyproteins can be found at GenBank Accession Nos. O55647, AAB24089, BAD51470, AAX62146, AAX62144, AAV28064, AAV28062, AAV28060, AAV28058, AAV28056, AAV28054, AAV28052, AAV28050, AAV28048, AAV28046, AAV28045, AAV28044, AAV28042, AAV28040, AAV28038, AAF80981, AAF80979, P36291, Q9IKB7, Q9IKB5, NP_{—}049359, and BAA24894. Some of these sequences vary slightly from each other, likely due to the fact that they are obtained from different viral isolates. However, they all contain 1135 amino acids and are all useful for the purpose of the present invention. The sequence from AAB24089 is provided in the sequence listing as SEQ ID NO:1 as an example. Amino acids 1-35 are a signal sequence (amino acid 35 is a peptidase cleavage site and amino acid 484 is putative peptidase site). G_{c}, also called G1, starts at amino acid 485 putatively and ends at amino acid 1135. G_{N}, also called G2, starts at amino acid 36 and ends at amino acid 484 putatively.

Both G_{N} and G_{c} have three domains: a domain that is outside of the viral membrane (receptor binding domain), a transmembrane domain, and a domain that is inside the viral membrane. The transmembrane domain is typically determined by its hydrophobicity. For example, different computer programs are available to assist such determination and can provide slightly different results. The hydrophilic sequence that is on the N-terminal side of the transmembrane domain is then identified as the receptor binding domain. For G_{N}, the receptor binding domain has been determined to start at amino acid 36 and ends anywhere from amino acid 308 to amino acid 345. It is noted that a lectin-like motif has been identified as amino acids 132-231 and an RGD motif (cell attachment site) has been identified as amino acids 41-43. One of these motifs or a functional fragment thereof may form the minimum sequence for binding to midgut receptor. For G_{c}, the receptor binding domain has been determined to start putatively at amino acid 485 and ends anywhere from amino acid 1050 to amino acid 1067.

Any fragment of an envelope/membrane glycoprotein that does not contain a significant portion of a hydrophobic domain such as a transmembrane domain is likely to be soluble. Examples of such soluble fragments include the extracellular domains or a fragment thereof. A fragment that contains an extracellular domain or a fragment thereof and fewer than six, five, four, three or two amino acids of the transmembrane domain is likely to be soluble.

In some embodiments, the genetic construct includes a first polynucleotide that encodes a soluble polypeptide containing the midgut receptor binding domain (or a functional fragment thereof) of an envelope/membrane glycoprotein. In other embodiments, the genetic construct includes a first polynucleotide that encodes G_{N} (SEQ ID NO:2) or G_{C} (SEQ ID NO:3) or a functional fragment thereof. By “functional fragment,” we mean that the fragment can bind to the midgut of the arthropod. In still other embodiments, the genetic construct includes a first polynucleotide that encodes G_{N-S} (SEQ ID NO:4), G_{C-S} or a functional fragment of either of the foregoing.

### Transgenic Plant.

The present invention also provides a transgenic plant, plant cell or plant tissue comprising a polynucleotide having a nucleotide sequence that encodes a midgut receptor binding domain (or a functional fragment thereof) of an envelope/membrane glycoprotein from a virus capable of infecting an arthropod operably linked to a plant-expressing promoter, wherein a polypeptide containing the midgut binding domain (or a functional fragment thereof) of the glycoprotein is expressed.

### Methods of Use.

The present invention also provides a method of reducing transmission of an arthropod-dependent viral plant disease. The method includes the step of exposing an arthropod to a polypeptide that contains a midgut receptor binding domain (or a functional fragment thereof) of an envelope/membrane glycoprotein from a virus capable of infecting the arthropod in an amount sufficient to reduce viral binding. Preferably, the polypeptide is a soluble polypeptide.

In some embodiments, the polypeptide further contains an active toxic fragment of a Bt toxin. In this regard, the polypeptide can also be used to kill the arthropod wherein the polypeptide including the active toxic fragment of the Bt toxin is applied to the arthropod in an amount sufficient to kill the arthropod. It is noted that other toxins (e.g., other insect toxins such as scorpion toxins) can be used to replace Bt toxin to practice the methods disclosed herein. It is further noted that it is well known in the art that the amino acids within the same conservative group can typically substitute for one another without substantially affecting the function of a protein. For the purpose of the present invention, such conservative groups are set forth in Table 1 below.

TABLE 1
Conservative substitution.

| | |
|---|---|
| Original Residue | Conservative Substitution |

| | |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

The present invention also provides a method of delivering an active toxic fragment of a Bacillus thuringinsis (Bt) toxin to an arthropod comprising the step of providing a polynucleotide that encodes a midgut receptor binding domain of an envelope/membrane glycoprotein from a virus capable of infecting the arthropod and a second polynucleotide that encodes an active toxic fragment of a Bacillus thuringinsis (Bt) toxin.

TSWV, like other members of the family Bunyaviridae, has a tripartite, negative-strand RNA genome (ambisense). The RNA encodes the following virus components: (1) a nucleocapsid protein from a small RNA segment, (2) two envelope/membrane glycoproteins from a medium RNA segment, and (3) a large protein from a large RNA segment. Of particular interest herein is the two envelope/membrane glycoproteins, which are derived from a polyprotein (SEQ ID NO:1) that is proteolytically processed to yield the two envelope/membrane glycoproteins, designated G_{N} (SEQ ID NO:2) and G_{C} (SEQ ID NO:3) based upon their position relative to the amino and carboxy termini of the polyprotein. Both G_{N} and G_{C} play a role in TSWV infection in its vectors.

Whitfield et al., (J. Virol. 78:13197-13206, 2004, which is herein incorporated by reference in its entirety) disclosed that TSWV acquisition by thrips depends upon a larval midgut receptor that binds the envelope/membrane glycoproteins of TSWV and that an exogenous, soluble G_{N} interfered with TSWV binding. However, it is not known based on the disclosure of Whitfield et al. whether reduced binding of TSWV can actually lead to reduced virus transmission because the smaller number of TSWV that bind to the midgut may produce enough viral particles so that viral transmission is not reduced. The inventors demonstrated here for the first time that the soluble G_{N} (G_{N-S}) can reduce viral transmission. This finding is quite surprising because one might expect that although G_{N-S} reduces binding and acquisition, a small number of virions may bind the midgut and enter the epithelial cells. Subsequent replication and virus spread to surrounding cells could overcome this initial reduction in binding and therefore transmission proceeds at usual frequency.

As used herein, “TSWV glycoproteins” means G_{N}, G_{C}, G_{N-S}, G_{C-S} and any functional fragment of these that retains an ability to bind to midgut receptors. By G_{N-S} (SEQ ID NO:4), we mean the soluble version of the G_{N} protein, which is known as a docking protein, meaning that it is involved in allowing the virus to attach to the surface of the cell to be entered. By G_{C-S}, we mean the soluble version of the G_{C} protein, which is involved in causing the virus to be internalized by the cell.

Although the examples below use F. occidentalis (i.e., western flower thrips) as the vector and TSWV as the virus, it is contemplated that other vector-virus combinations could be used in the practice of the invention, such as those described in Table 2. That is, viruses have envelope/membrane glycoproteins recognized by receptors in their respective vector(s). Therefore, TSWV transmission by thrips has many features in common with a large number of insect-transmitted Bunyaviruses and Rhabdoviruses, including important pathogens of humans and other animals that may be exploited in a similar manner as that described below.

TABLE 2
Virus-Vector Combinations

| Tospovirus species | Thrips vectors |
|---|---|
| Chrysanthemum stem necrosis virus | Frankliniella occidentalis |
| | F. schultzei |
| Groundnut bud necrosis virus | F. schultzei Thrips palmi |
| | Scirtothrips dorsalis |
| Groundnut chlorotic fanspot virus | S. dorsalis |
| Groundnut ringspot virus | F. occidentalis F. schultzei |
| Groundnut yellow spot virus | S. dorsalis |
| Impatiens necrotic spot virus | F. occidentalis |
| Iris yellow spot virus | T. tabaci |
| Physalis severe mottle virus | T. palmi |
| Tomato chlorotic spot virus | F. intonsa |
| | F. occidentalis |
| | F. schultzei |
| Tomato spotted wilt virus | F. bispinosa |
| | F. fusca |
| | F. intonsa |
| | F. occidentalis |
| | F. schultzei |
| | T. setosus |
| | T. tabaci |
| Watermelon bud necrosis virus | T. palm |
| Watermelon silver mottle virus | T. palmi |
| Zucchini lethal chlorotic virus | F. zucchini |

### Biopesticidal Composition.

In an alternate embodiment, the present invention provides a biopesticidal composition for preventing transmission of an arthropod-dependent viral plant disease. By “biopesticidal composition” we mean a composition used to control and/or prevent transmission of an arthropod-dependent viral plant disease such as the virus-vector combinations described in Table 2. The biopesticidal composition, or “bait”, of the present invention provides the arthropod with an envelope/membrane glycoprotein from a virus capable of infecting the arthropod. In a preferred embodiment, the purified G_{N-S} is fused with Bt and mixed with pollen, wherein arthropods such as thrips ingest the pollen and subsequently die.

The composition comprises an effective amount of an envelope/membrane glycoprotein from a virus capable of infecting the arthropod and optionally includes an active toxic fragment of a Bacillus thuringinsis (Bt) toxin, with or without an agriculturally acceptable carrier. The agriculturally acceptable carrier may be in the form of a liquid, powder, granules or small particles known in the art. Solid and liquid formulations may be used. Additional expedients used in the art, such as emulsifiers, thickeners, foaming agents, etc., may be used. The composition may also contain other chemical or biological control agents. The composition may also be applied, either simultaneously or sequentially, with other chemical or biological control agents. Application of the composition may be accomplished using standard operating equipment used in the agricultural or horticultural industry, for example by conventional ground spreaders or sprayers or aerially.

Those working in this field would of course be readily able to determine in an empirical manner (based on the teaching of this application) the optimum rates of application for any given target organisms to be killed or eliminated. The amount of biopesticidal composition used will be at least an effective amount to reduce insect pests. The term “effective amount,” as used herein, means the minimum amount of the biopesticidal composition needed to kill the target insects. The precise amount of the biopesticidal composition can easily be determined by one skilled in the art given the teaching of this application. The concentration used in the composition of the present invention is readily determinable by skilled practitioners depending, for example, on the extent and degree of infestation, time, weather conditions, life cycle of the pest, and concurrent use of other insecticides. Those working in this field would of course be readily able to determine in an empirical manner (based on the teaching of this application) the optimum timing of application for any given combination of target organisms to be killed or eliminated.

### Kits.

In an alternate embodiment of the invention, a kit for preparing the biopesticidal composition of the present invention is provided. In one embodiment, the kit comprises a genetic construct according to the present invention, and instructions for use. Optionally the kit may include an agriculturally-acceptable carrier.

By “instructions for use” we mean a publication, a recording, a diagram, or any other medium of expression which is used to communicate the usefulness of the invention for one of the purposes set forth herein. The instructional material of the kit can, for example, be affixed to a container which contains the present invention or be shipped together with a container which contains the invention. Alternatively, the instructional material can be shipped separately from the container or provided on an electronically accessible form on a internet website with the intention that the instructional material and the biocompatible hydrogel be used cooperatively by the recipient.

The invention will be more fully understood upon consideration of the following non-limiting Examples. The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Example 1

### TWSV Glycoprotein and Fragments Thereof for Prevention of Insect Acquisition and Subsequent Insect Transmission of a Virus

In this example, the inventors determine if G_{N-S} alters TSWV transmission by thrips and, if so, the duration of this effect. Insects were given an acquisition access period (AAP) with G_{N-S} mixed with purified virus and individual insects were assayed for transmission. The inventors found that G_{N-S} significantly reduced the percent of transmitting adults by eight-fold. In a second study, thrips were given an AAP on G_{N-S} protein and then placed on TSWV-infected plant material. Individual insects were assayed for transmission over three time intervals of 2-3, 4-5, and 6-7 days post pupal eclosion.

The inventors observed a significant reduction in virus transmission and the inhibition of transmission persisted to the same degree throughout the time course. Real time-RT-PCR analysis of virus titer in individual insects revealed that the proportion of thrips infected with virus was reduced three-fold when insects were pre-exposed to the G_{N-S} protein as compared to no exposure to protein, and non-transmitters were not infected with virus. These results demonstrate that thrips transmission of a tospovirus can be reduced by exogenous viral glycoprotein. The findings disclosed here are quite surprising because one might expect that although G_{N-S} reduces binding and acquisition, a small number of virions may bind the midgut and enter the epithelial cells. Subsequent replication and virus spread to surrounding cells could overcome this initial reduction in binding and therefore transmission proceeds at usual frequency. The inventors found that this was not the case.

Constructs of TSWV G_{N} and G_{C} glycoproteins, as well as truncated G_{N-S} and G_{C-S} glycoproteins (i.e., TSWV glycoproteins), are created using methods described in Whitfield et al., supra. Where necessary, truncated TSWV glycoproteins are tested first for an ability to prevent insect acquisition as described in Whitfield et al., supra. If a TSWV glycoprotein or truncated fragment thereof is shown to prevent insect acquisition, it may therefore be used to prevent insect transmission as described herein.

F. occidentalis cultures. A colony of F. occidentalis is maintained on green bean pods (Phaseolus vulgaris) as described by Ullman et al., “A midgut barrier to tomato spotted wilt virus acquisition by adult western flower thrips,” Phytopathology 82:1333-1342 (1992).

To generate first instar larvae for transmission experiments, beans are incubated with adult thrips for three days to allow the insects to oviposit. Thrips are removed and the beans are incubated at 23° C. for 24 hours. Larval thrips (0-24 hours old) are harvested and pooled. Aliquots of the same thrips cohort are transferred to feeding chambers for transmission experiments.

TSWV purification. TSWV (isolate TSWV-MT2) are maintained by thrips transmission and are mechanically transferred only one time after thrips transmission to maintain thrips-transmissibility of the isolate. TSWV-infected Datura stramonium leaves used for virus purification are harvested two weeks post-inoculation, and TSWV virions are isolated by a modified and shortened version of the method described in Gonsalves & Trujillo. Gonsalves D & Trujillo E., “Tomato spotted wilt virus in papaya and detection of the virus by ELISA,” Plant Dis. 70:501-506 (1986).

Briefly, a triturated and filtered homogenate is centrifuged at 8,000×g for 15 minutes. The supernatant is removed, 50 ml of 10 mM sodium sulfite is added, and pellets are re-suspended by stirring on ice at 4° C. for 95 minutes. The virus is centrifuged at 4° C. for 20 minutes at 8,600×g and the supernatant collected. The supernatant is centrifuged for 39 minutes at 88,000×g. The supernatant is removed, and the pellet is re-suspended in 500 μl sodium sulfite. Before virus is presented to thrips, protease inhibitors (1 μg/ml each of antipain, aprotinin, chymostatin, leupeptin and pepstatin) are added to the virus solution. Virus viability is tested by mechanically inoculating Nicotiana glutinosa, a local-lesion host.

Concomitant feeding experiments with soluble TSWV glycoproteins or fragments thereof and TSWV. To assay for the ability of soluble TSWV glycoproteins to inhibit TSWV transmission, first instar larval thrips are given an acquisition access period (AAP) on solutions containing purified TSWV or purified TSWV combined with the soluble TSWV glycoprotein. By way of example, the TSWV glycoprotein can be G_{N-S} (i.e., amino acids 35 to 309 of SEQ ID NO:1) as described in Whitfield et al.

Briefly, thrips are fed in cylindrical 25-mm-diameter containers similar to those described in Hunter et al., “A novel method for tospovirus acquisition by thrips,” Phytopathology 85:480-483 (1995). Ends of the tubes are sealed with a thin layer of Parafilm®, which allows the insects to feed. The feeding solutions are as follows: (1) TF buffer (PBS, 10% glycerol, 0.01% Chicago sky blue and 5 mg/ml BSA); (2) soluble TSWV glycoprotein (0.1 nM)+TSWV in TF buffer; or (3) TSWV in TF buffer. Insects are fed 0.1 nM soluble TSWV glycoprotein because this concentration was previously shown to bind larval thrips midguts and inhibit TSWV acquisition as described in Whitfield et al., supra.

Purified virus solutions are diluted to the same concentrations, and the methods of Whitfield et al., supra, are used to manipulate the thrips. Briefly, 100 μl of feeding solution is sandwiched between two layers of Parafilm®. Thrips are allowed to feed on the solutions for 16 hours and then transferred to beans to be reared to adulthood. Thirteen days after the AAP, the adult insects are moved to D. stramonium leaf discs held in 1.5 ml microfuge tubes. After allowing the thrips to feed for six days, the leaf discs are moved to 24-well plates with 1 ml of deionized water per well. Leaf discs are incubated at room temperature for three days, ground in a plant leaf grinder, and assayed for virus infection using a QTA Tospo ELISA kit (Agdia; Elkhart, Ind.).

Sequential feeding experiments with soluble TSWV glycoproteins and TSWV. Larval thrips are given a two-hour AAP with a soluble TSWV glycoprotein (0.1 nM in TF buffer) or TF buffer as described for in vivo binding assays in Whitfield et al., supra. Thrips are then placed on a bouquet of TSWV-infected D. stramonium leaves for three hours. Insects are then transferred to bean pods and reared to adulthood. After adult emergence, individual thrips are placed on D. stramonium leaf discs for 48 hour-inoculation access periods (IAPs) and discs are replaced three times (2-3, 4-5, and 6-7 days post adult-eclosion) in order to assess thrips transmission over time. Leaf discs are incubated in water for four days and TSWV detected by DAS-ELISA as noted above.

Real time RT-PCR analysis of TSWV in thrips. To determine infection status (incidence and titer), individual insects are fed either soluble TSWV glycoprotein followed by an AAP on TSWV-infected D. stramonium leaves, and then a 2-3 day IAP period on D. stramonium leaf discs. Twenty insects from each treatment (soluble TSWV glycoprotein+TSWV and TSWV alone) are sampled from their respective leaf discs.

RNA extraction is performed on individual insects as described in Boonham et al. Boonham N. et al., “Detection of tomato spotted wilt virus (TSWV) in individual thrips using real time fluorescent RT-PCR (Taqman),” J. Virol. Meth. 101:37-48 (2002). cDNA is made using the iScript cDNA synthesis kit (Bio-Rad; Hercules, Calif.), and 15 μl of RNA is used for each 20 μl reaction. Real-time RT-PCR is performed using a iCycler iQ Thermal Cycler with 96×0.2 ml reaction module and iCycler iQ software (Bio-Rad). F. occidentalis actin primers are used for amplification of the actin gene (forward primer 5′GGTATCGTCCTGGACTCTGGTG 3′ (SEQ ID NO:5); reverse primer 5′ GGGAAGGGCGTAACCTTCA 3′ (SEQ ID NO:6)). Boonham et al., supra. Primers to the TSWV nucleocapsid (N) gene are used for assaying virus (forward primer ′ GCTTCCCACCCTTTGATTC 3′ (SEQ ID NO:7); reverse primer 5′ ATAGCCAAGACAACACTGATC 3′ (SEQ ID NO:8)). iQ SYBR Green (BioRad) is used for all RT-PCR reactions according to manufacturer's specifications.

Briefly, reactions are performed in a volume of 20 μl, using iQ SYBR Green Supermix, 20 pmol of forward and reverse primers, and the same volume of cDNA for each reaction. Reactions are performed in the iCycler using a two-step amplification plus melting curve protocol. TSWV N gene expression (target) is normalized to actin expression (internal reference) to calculate the relative abundance of TSWV N RNA in each insect using the inverse equation in Pfaffl: E_{actin}^{Ct(actins)}/E_{N}^{Ct(N)}; where E=PCR efficiency of a primer pair (actin or N). Pfaffl M, “A new mathematical model for relative quantification in real-time RT-PCR,” Nucl. Acids Res. 29:e45 (2001). The proportion of TSWV-infected insects is calculated for each treatment.

Local lesion assay. Soluble TSWV glycoprotein, such as G_{N}-S or a fragment thereof, is mixed with purified TSWV and rub-inoculated onto local lesion plant hosts, such as N. glutinosa, to determine if it affects virus viability (measured by infectivity of plant host tissue). Virus is purified as described and mixed with the soluble TSWV glycoprotein in PBS, pH 7.4 or PBS. A small amount of celite (0.05 g) is added to act as an abrasive, and the protein-virus mix is diluted in 0.01 M sodium sulfite. The solutions are rub-inoculated onto leaves using a cotton-tipped applicator. Local lesions are counted three days post-inoculation.

Soluble TSWV glycoprotein reduces TSWV transmission when insects are fed concomitantly with purified TSWV. Insects given an AAP on purified TSWV transmit it to D. stramonium leaf discs. In contrast, insects given an AAP on TSWV+soluble glycoprotein show a reduction in the proportion of transmitting adults when compared to insects fed on TSWV alone. Transmitting insects given an AAP on a buffer alone (not exposed to TSWV) are not significantly different from insects exposed to the soluble TSWV glycoprotein+TSWV mixture. As such, soluble TSWV glycoprotein inhibition of acquisition reduces the number of insects capable of transmitting the virus to host plants.

Soluble TSWV glycoprotein reduces TSWV transmission even when insects are fed purified protein prior to AAP on TSWV-infected plant tissue. The incidence of transmitting insects fed TSWV alone is significantly different when compared to insects sequentially fed soluble TSWV glycoprotein and TSWV and at all times tested. Insects fed on a BSA-buffer solution transmit TSWV up to the 5-day IAP. In contrast, insects given the soluble TSWV glycoprotein solution prior to AAP on TSWV-infected leaves are less likely to transmit virus. Consequently, inhibition of acquisition through pre-feeding on soluble TSWV glycoprotein effectively reduces TSWV transmission.

Virus infection status of transmitting and non-transmitting thrips. The proportion of thrips infected with virus is reduced when insects are predisposed to the soluble TSWV glycoprotein as compared to insects that had no exposure to the soluble TSWV glycoprotein.

Local lesion assay. The number of local lesions per leaf is the same for the soluble TSWV glycoprotein+TSWV and TSWV alone treatments. As such, soluble TSWV glycoprotein does not affect the infectivity of purified virus. Therefore, any observed inhibition of thrips virus acquisition and the resulting reduced transmission are likely due to interference with the ability of TSWV glycoproteins to bind a cell receptor(s) in the thrips midgut.

As an agent for preventing virus transmission, any suitable method of treating a plant, plant part or seed with the soluble TSWV glycoproteins can be used in the present invention. Preferably, a plant, plant part or seed is treated with a solution that contains soluble TSWV glycoproteins. The preferred solvent for soluble TSWV glycoproteins for purpose of the present invention is water. However, other suitable solvents such as organic solvents can also be used. To treat a plant, plant part or seed with a solution soluble TSWV glycoproteins, the plant, plant part or seed can be sprayed with the solution, or it can be dipped or soaked in the solution. Other suitable methods of exposing a plant, plant part or seed, and ultimately an arthropod, to soluble TSWV glycoproteins can also be used. For example, granules or powders containing soluble TSWV glycoproteins can be applied on the ground near plants susceptible to TSWV such that TSWV glycoproteins are either ingested by the arthropod or are taken up by the plant and subsequently ingested by the arthropod.

### Example 2

### (Prophetic): Soluble TSWV Glycoproteins and Fragments Thereof as an Insecticide when Fused with a Polypeptide that Contains an Active Toxic Fragment

In this example, the inventors determine the minimum G_{N} and G_{c} fragments capable of binding to TSWV vector midgut epithelium that effectively blocks virus transmission are determined by producing truncated versions the TSWV surface glycoproteins. The resulting, purified peptides are tested in the inventors' established in vitro transmission assay. A fusion between a minimal transmission inhibiting peptide (TIP) and selected constructs of Bacillus thuringinsis toxin is made. The effectiveness of the fusion construct as a means to control thrips in an experimental arena containing host plants is then tested.

Using the G_{N-S}, G_{C-S} and various fragments described above (i.e., minimal transmission inhibiting peptide (TIP)) one can make a genetic construct that encodes a fusion protein having a midgut receptor binding domain and an active toxic fragment of a Bacillus thuringinsis (Bt) toxin. The family of genes coding for the Bt toxin is known as the Cry gene family. See U.S. Pat. No. 5,888,801; and EP Patent No. 1,356,054. Bt toxins offer one alternative to chemical pesticides, since Bt toxins are non-toxic to vertebrates (generally Bt toxin targets insects within a single order) and are more benign to the environment. Different strains of Bt are specific to different receptors in insect gut wall tissues. For example, Bt toxins have specific activities against species of the orders Lepidoptera (moths/butterflies), Diptera (flies/mosquitoes) and Coleoptera (beetles). Alternatively, other toxins suitable for the present invention include Photorhabdus luminescens toxin. See Blackburn M, et al., “A novel insecticidal toxin from Photorhabdus luminescens, toxin complex a (Tca), and its histopathological effects on the midgut of Manduca sexta,” Appl. Environ. Microbiol. 64: 3036-3041 (1998); and U.S. Pat. No. 7,030,296.

Typically, Bt toxins are synthesized as protoxins during sporulation of Bt strains and are deposited in the parasporal crystal. On ingestion by insect larvae, the crystals are solubilized in an alkaline environment of the insect gut. Subsequently, insect proteinases release an active toxic fragment, which binds to specific receptors on the midgut cells of susceptible larvae and causes formation of pores, leading in turn to colloid osmotic lysis of the cell. The toxicity of the activated Bt toxin, however, is dependent on the presence of specific receptor sites on the insect's gut wall. Therefore, if an active toxic fragment of a Bt toxin is fused with any of the soluble TSWV glycoproteins described herein, thrips may become sensitive to Bt.

To change the specificity of a Bt toxin, clones will be provided and PCR primers will be designed to overlap the soluble TSWV glycoprotein identified above in such a way as to remove a wild-type Bt ligand domain and replace it with the soluble TSWV receptor ligand domains identified above. This will create a thrips-specific moiety by targeting the Bt specifically to thrips midgut receptors instead of the Bt receptor.

The fusion proteins can be tested by feeding it directly to first and second instar larva and adult thrips in vitro using the methods described above. Control experiments will involve feeding the wild-type Bt toxin to insects susceptible to the toxin and feeding the engineered construct to the same susceptible insects.

Methods of constructing recombinant nucleic acid sequences that encode fusion proteins are well-known to one skilled in the art. See, e.g., Carson S & Robertson D, “Manipulation and expression of recombinant DNA,” (2nd ed. 2005); IJkel W. et al., “A novel baculovirus envelope fusion protein with a proprotein convertase cleavage site,” Virology 275:30-41 (2000); Mehlo L. et al., “An alternative strategy for sustainable pest resistance in genetically enhanced crops,” Proc. Natl. Acad. Sci. USA 102:7812-7816 (2005); and U.S. Pat. No. 7,214,788, each of which is incorporated herein by reference as if set forth in its entirety.

As an insecticide, any suitable method of treating a plant, plant part or seed with the fusion protein can be used in the present invention. The fusion protein simply need to be available for larvae to ingest. Preferably, a plant, plant part or seed is treated with a solution that contains a fusion protein that is soluble. The preferred solvent for soluble fusion proteins for purpose of the present invention is water. However, other suitable solvents such as organic solvents can also be used. To treat a plant, plant part or seed with a solution of soluble proteins, the plant, plant part or seed can be sprayed with the solution, or it can be dipped or soaked in the solution. Other suitable methods of exposing a plant, plant part or seed to the soluble fusion proteins can also be used. For example, granules or powders containing the fusion protein can be applied on the ground near plants susceptible to TSWV.

### Example 3

### (Prophetic): Transgenic Plants Expressing Soluble TSWV Glycoproteins and Fragments Thereof

To make a transgenic plant or plant part that expresses a transgene, one needs to make a genetic construct capable of expressing the polynucleotide in the plant. One also needs a method to insert the genetic construction into the plant.

The tools and techniques for making genetic constructs that express proteins in plants are well known to one skilled in the art. Any genetic construct intended to cause the synthesis in the cells of the plant of a polypeptide or protein must include a sequence of DNA (i.e., a polynucleotide that can be genomic DNA or cDNA) that specifies the sequence of the polypeptide or protein to be produced in the resultant plant. For a protein coding sequence to be expressed in a plant to produce a polypeptide or protein, it must be placed under the control of a plant expressible promoter and be followed by a plant transcriptional terminator sequence, also known as a polyadenlyation sequence. The plant expressible promoter is a promoter that will work in plants, usually either of plant origin or from a plant pathogen like a virus (e.g., Cauliflower mosaic virus) or a bacteria (e.g., Agrobacterium promoters like the nopaline synthase promoter).

Plant promoters from pathogens tend to be constitutive promoters, meaning that they actually express the transgene in all of the tissues of the plant at all times. Examples of constitutive promoters useful in plant genetic constructions include, without limitation, the 35S RNA and 19S RNA promoters of the Cauliflower mosaic virus (Brisson N. et al., “Expression of a bacterial gene in plants by using a viral vector,” Nature, 310:511-514 (1984)), and the opine synthase promoters carried on the tumor-inducing plasmids of Agrobacterium tumefaciens such as the nopaline synthase promoter (Ebert P. et al., “Identification of an essential upstream element in the nopaline synthase promoter by stable and transient assays,” PNAS 84:5745-5749 (1987)) and the mannopine synthase promoter (Velten J. et al., “Isolation of a dual plant promoter fragment from the Ti plasmid of Agrobacterium tumefaciens,” EMBO J. 3:2723-2730 (1984)).

Other plant promoters are known to be tissue specific or developmentally specific, while others are intended to be inducible (e.g., heat shock or metal ion induced promoters). An example of tissue specific promoters is the maize γ-zein promoter. Examples of inducible promoters include, but are not limited to, heat shock promoters such as soybean hsp17.5E or hsp17.3 (Gurley W. et al., “Upstream sequences required for efficient expression of a soybean heat shock gene,” Mol. Cell Biol. 6:559-565 (1986)), light-regulated promoters such as the promoter for the small subunit or ribulose bisphosphate carboxylase (ssRUBISCO) (Coruzzi G, et al., “Tissue-specific and light-regulated expression of a pea nuclear gene encoding the small subunit of ribulose-1,5-bisphosphate carboxylase,” EMBO J. 3:1671-1679 (1984); Broglie R, et al., “Light-regulated expression of a pea ribulose-1,5-bisphosphate carboxylase small subunit gene in transformed plant cells,” Science 224:838-843 (1984)), chemical-regulated promoters such as Maize In2-1 and 2-2 that are regulated by benzenesulfonamides, e.g., herbicide safeners (Hershey H & Stoner T, “Isolation and characterization of cDNA clones for RNA species induced by substituted benzenesulfonamides in corn,” Plant Mol. Biol. 17:679-690 (1991)), and alcA and alcR promoter/transcription factor system that is induced by the application of ethanol (Caddick M, et al., “An ethanol inducible gene switch for plants used to manipulate carbon metabolism,” Nat. Biotech. 16:177-180 (1998)).

Any of the promoters described above may be used in the practice of this invention depending on the intended effect on the transgenic plant to be produced. For example, adjusting the expression level of a polynucleotide encoding a non-plant transgene by varying promoter strength may determine the likelihood of the transgenic plant to have the varying degrees of protection from plant viruses.

Optionally, a selectable marker may be associated with a genetic construct used to generate a transgenic plant. As used herein, the term “marker” refers to a gene encoding a trait or a phenotype which permits the selection of, or the screening for, a plant or plant cell containing the marker. Preferably, the marker is an antibiotic resistance gene, whereby the appropriate antibiotic can be used to select for transformed cells from among cells that are not transformed. Examples of suitable selectable markers include adenosine deaminase, dihydrofolate reductase, hygromycin-B-phosphotransferase, thymidine kinase, xanthine-guanine phospho-ribosyltransferase, and amino-glycoside 3′-O-phosphotransferase II (which confers kanamycin, neomycin and G418 resistance). Other suitable markers will be known to one of skill in the art.

Several methods have been demonstrated to insert genes into plants to make them transgenic. The most widely used methods, broadly defined, are Agrobacterium-mediated transformation and accelerated particle mediated transformation. The various techniques of Agrobacterium-mediated plant transformation make use of the natural ability of the plant pathogens of the Agrobacterium genus to transfer DNA from a plasmid in the bacteria into the genome of a plant cell.

Particle-mediated plant transformation techniques utilize DNA-coated small carrier particles accelerated from a device, often referred to as a gene gun, into the cells of a plant. The full implementation of either approach requires techniques to recover a fully mature, morphologically normal plant from the transformed cells. The techniques often therefore involve either selection or screening protocols to identify which plant cell was transformed and regeneration protocols to recover a whole plant from a single transformed plant cell. As mentioned above, these techniques have been worked out for many plant species and many, and perhaps all, of the economically important plant species.

Viruses such as the Cauliflower mosaic virus (CaMV) may also be used as a vector for introducing a transgene into plant cells (U.S. Pat. No. 4,407,956). The CaMV viral DNA genome is inserted into a parent bacterial plasmid creating a recombinant DNA molecule which can be propagated in bacteria. After cloning, the recombinant plasmid again may be cloned and further modified by introduction of the desired polynucleotide sequence. The modified viral portion of the recombinant plasmid is then excised from the parent bacterial plasmid, and used to inoculate the plant cells or plants.

Other techniques, such as electroporation have also been used to make transgenic plants. But fundamentally for the invention disclosed here, the particular technique of plant transformation does not matter. Once the plant has been genetically engineered, and a transgenic plant has been created, the method of transformation of the original plant becomes irrelevant.

A transgene inserted into the genome of one plant is then fully inheritable by progeny plants of the original genetically engineered plant by normal rules of classical plant breeding. For example, in vegetatively propagated crops, the mature transgenic plants are propagated by the taking of cuttings or by tissue culture techniques to produce multiple identical plants. Selection of desirable transformed plants is made and new varieties are obtained and propagated vegetatively for commercial use. In seed-propagated crops, the mature transgenic plants can be self crossed to produce a homozygous inbred plant. The inbred plant produces seed containing the newly introduced transgene. These seeds can be grown to produce plants that would produce the selected phenotype.

It should be understood that techniques of plant genetic engineering have been developed to the point where it is now practical to place any genetic construct into almost any useful plant species. The process does, however, still involve some random processes, most notably that insertions of foreign DNA into the genome of plants still occurs at random sites in the plant genome. As a result, in any group of plants emerging from a plant transformation process, the results achieved for the different gene insertion events will vary, sometimes dramatically, depending on where the transgene is inserted. However, this variation does not mean stable results cannot be achieved, since the results tend to be consistent generation-to-generation for each specific genetic insertion. One can also take advantage of this variation to generate lines with cornstarch characteristics changed to different degrees.

A transgenic plant therefore would be engineered to harbor a polynucleotide that encodes a midgut receptor binding domain of an envelope/membrane glycoprotein from a virus capable of infecting the arthropod or a fusion protein discussed above, under the control of a plant-expressible promoter.

It should be noted that the above description, attached figures and their descriptions are intended to be illustrative and not limiting of this invention. Many themes and variations of this invention will be suggested to one skilled in this and, in light of the disclosure. All such themes and variations are within the contemplation hereof. For instance, while this invention has been described in conjunction with the various exemplary embodiments outlined above, various alternatives, modifications, variations, improvements, and/or substantial equivalents, whether known or that rare or may be presently unforeseen, may become apparent to those having at least ordinary skill in the art. Various changes may be made without departing from the spirit and scope of the invention. Therefore, the invention is intended to embrace all known or later-developed alternatives, modifications, variations, improvements, and/or substantial equivalents of these exemplary embodiments.

We claim:

1. A genetic construct for inhibiting virus transmission by an arthropod comprising a first polynucleotide that encodes a midgut receptor binding domain of an envelope/membrane glycoprotein from a virus capable of infecting the arthropod and a second polynucleotide that encodes an active toxic fragment of a Bacillus thuringinsis (Bt) toxin.

2. A genetic construct as recited in claim 1, further comprising a promoter operably linked to the first and second polynucleotides for expressing a fusion protein.

3. The genetic construct as recited in claim 1, wherein the arthropod is a member of Thysanoptera.

4. The genetic construct as recited in claim 3, wherein the arthropod is a thrips.

5. The genetic construct as recited in claim 1, wherein the virus is dependent upon a member of Thysanoptera for transmission to a host plant.

6. The genetic construct as recited in claim 5, wherein the virus is tomato spotted wilt virus.

7. The genetic construct as recited in claim 1, wherein the nucleotide sequence for the envelope/membrane glycoprotein is selected from nucleotide sequences that encode amino acid sequences for G_{N} (SEQ ID NO:2), G_{C} (SEQ ID NO:3) or a functional fragment of the foregoing.

8. The genetic construct as recited in claim 7, wherein the nucleotide sequence for the envelope/membrane glycoprotein is selected from nucleotide sequences that encodes an amino acid sequence for G_{N}-S(SEQ ID NO:4), G_{C}-S or a functional fragment of the foregoing.

9. A transgenic plant, plant cell or plant tissue comprising a nucleic acid sequence encoding an envelope/membrane glycoprotein or functional fragment thereof from a virus capable of infecting an arthropod operably linked to a plant-expressing promoter.

10. The transgenic plant, plant cell or plant tissue as recited in claim 9, wherein the arthropod is a member of Thysanoptera.

11. The transgenic plant, plant cell or plant tissue as recited in claim 10, wherein the arthropod is a thrips.

12. The transgenic plant, plant cell or plant tissue as recited in claim 9, wherein the virus is dependent upon a member of Thysanoptera for transmission to a host plant.

13. The transgenic plant, plant cell or plant tissue as recited in claim 12, wherein the virus is tomato spotted wilt virus.

14. The plant tissue as recited in claim 9, wherein the tissue is a seed.

15. The transgenic plant, plant cell or plant tissue as recited in claim 9, wherein the nucleic acid soluble envelope/membrane glycoprotein is G_{N} (SEQ ID NO:2) and G_{C} (SEQ ID NO:3) or a fragment of the foregoing.

16. The transgenic plant, plant cell or plant tissue as recited in claim 9, wherein the nucleic acid sequence encodes G_{N-S} (SEQ ID NO:4) G_{C-S} or a functional fragment of the foregoing.

17. The transgenic plant, plant cell or plant tissue as recited in claim 9, wherein the nucleic acid sequences further comprising an active toxic fragment of a Bt toxin.

18. A method of preventing transmission of an arthropod-dependent viral plant disease comprising the step of exposing an arthropod to a polypeptide that contains a midgut receptor binding domain of an envelope/membrane glycoprotein from a virus capable of infecting the arthropod in an amount sufficient to reduce viral binding.

19. The method as recited in claim 18, wherein the polypeptide in provided in a solution or suspension and applied to plants or plant parts that the arthropod feeds upon.

20. The method as recited in claim 18, wherein the polypeptide is provided in a transgenic plant.

21. The method as recited in claim 19, wherein the soluble, envelope/membrane glycoprotein is in a transgenic plant.

22. The method as recited in claim 18, wherein the soluble, envelope/membrane glycoprotein is G_{N} (SEQ ID NO:2), G_{C} (SEQ ID NO:3) or a functional fragment of the foregoing.

23. The method as recited in claim 18, wherein the soluble, envelope/membrane glycoprotein is G_{N-S} (SEQ ID NO:4), G_{C-S} or a functional fragment of the foregoing.

24. The method as recited in claim 18, wherein the soluble, envelope/membrane glycoprotein further comprises an active toxic fragment of a Bt toxin.

25. A method of delivering an active toxic fragment of a Bacillus thuringinsis (Bt) toxin to an arthropod comprising the step of providing a polynucleotide that encodes a midgut receptor binding domain of an envelope/membrane glycoprotein from a virus capable of infecting the arthropod and a second polynucleotide that encodes an active toxic fragment of a Bacillus thuringinsis (Bt) toxin.

26. The method as recited in claim 25, wherein the arthropod is a member of Thysanoptera.

27. The method as recited in claim 26, wherein the arthropod is a thrips.

28. The method construct as recited in claim 25, wherein the virus is dependent upon a member of Thysanoptera for transmission to a host plant.

29. The method construct as recited in claim 25, wherein the virus is tomato spotted wilt virus.

30. A biopesticidal composition for preventing transmission of an arthropod-dependent viral plant disease comprising a genetic construct comprising a first polynucleotide that encodes a midgut receptor binding domain of an envelope/membrane glycoprotein from a virus capable of infecting the arthropod.

31. The biopesticidal composition of claim 31 additionally comprising a second polynucleotide that encodes an active toxic fragment of a Bacillus thuringinsis (Bt) toxin.


SEQUENCE LISTING (8 sequences)

SEQ ID NO: 1 - protein, 1135 aa, Tomato spotted wilt virus
MRILKLLELVVKVSLFTIALSSVLLAFLIFRATDAKVEIIRGDHPEIYDDSAENEVPTAASIQREAILETLTNLMLESRTPGTRQIREEKSTIPISAEPTTQKTISVLDLPNNCLNASSLKCEIKGISTYNVYYQVENNGVIYSCVSDSAEGLEKCDNSLNLPKRFSKVPVIPITKLDKKRHFSVGGKFFISESLTQDNYPITYNSYPTNGTVSLQTVKLSGDCKITKSNFANPYTVSITSPEKIMGYLIKKPGENVEHKVISFSGSASITFTEEMLDGEHNLLCGDKSAKIPKTNKRVRDCIIKYSKSIYKQTACINFSWIRLILIALLIYFPIRWLVNKTTKPLFLWYDLMGLITYPVLLLINCLWKYFPLKCSNCGNLCIVTHECTKVCICNKSKASKEHSSECPILSKEADHDYNKHKWTSMEWFHLIVNTKLSLSLLKFVTEILIGLVILSQMPMSMAQTTQCLSGCFYVPGCPFLVTSKFEKCSEKDQCYCNVKEDKIIESIFGTNIVIEGPNDCIENQNCIARPSIDNLIKCRLGCEYLDLFRNKPLYNGFSDYTGSSLGLTSVGLYEAKRLRNGIIDSYNRQGKISGMVAGDSLNKNETSIPENILPRQSLIFDSVVDGKYRYMIEQSLLGGGGTIFMLNDKTSETAKKFVIYIKSVGIHYEVSEKYTTAPIQSTHTDFYSTCTGNCDTCRKNQALTGFQDFCVTPTSYWGCEEAWCFAINEGATCGFCRNIYDMDKSYRIYSVLKSTIVADVCISGILGGQCSRITEEVPYENTLFQADIQADLHNDGITIGELIAHGPDSHIYSGNIANLNDPVKMFGHPQLTHDGVPIFTKKTLEGDDMSWDCAAIGKKSVTIKTCGYDTYRFRSGLEQISDIPVSFKDFSSFFLAKSFSLGKLKMVVDLPSDLFKVAPKKPSITSTSLNCNGCLLCGQGLSCLLEFFSDLTFSTAISIDACSLSTYQLAVKKGSNKYNITMFCSANPDKKKMTLYPEGNPDISVEVLVNNVIVEEPENIIDQNDEYAHEEQQYNSDSSAWGFWDYIKSPFNFIASYFGSFFDTIRVVLLIAFIFLVTYFCSILTSICKGYVKNESYKSRSKIEDDDEPEIKAPMLMKDTMTRRRPPMDFSHLV

SEQ ID NO: 2 - protein, 459 aa, Tomato spotted wilt virus
KVEIIRGDHPEIYDDSAENEVPTAASIQREAILETLTNLMLESRTPGTRQIREEKSTIPISAEPTTQKTISVLDLPNNCLNASSLKCEIKGISTYNVYYQVENNGVIYSCVSDSAEGLEKCDNSLNLPKRFSKVPVIPITKLDKKRHFSVGGKFFISESLTQDNYPITYNSYPTNGTVSLQTVKLSGDCKITKSNFANPYTVSITSPEKIMGYLIKKPGENVEHKVISFSGSASITFTEEMLDGEHNLLCGDKSAKIPKTNKRVRDCIIKYSKSIYKQTACINFSWIRLILIALLIYFPIRWLVNKTTKPLFLWYDLMGLITYPVLLLINCLWKYFPLKCSNCGNLCIVTHECTKVCICNKSKASKEHSSECPILSKEADHDYNKHKWTSMEWFHLIVNTKLSLSLLKFVTEILIGLVILSQMPMSMAQTTQCLSGCFYVPGCPFLVTSKFEKCSEKDQ

SEQ ID NO: 3 - protein, 641 aa, Tomato spotted wilt virus
CYCNVKEDKIIESIFGTNIVIEGPNDCIENQNCIARPSIDNLIKCRLGCEYLDLFRNKPLYNGFSDYTGSSLGLTSVGLYEAKRLRNGIIDSYNRQGKISGMVAGDSLNKNETSIPENILPRQSLIFDSVVDGKYRYMIEQSLLGGGGTIFMLNDKTSETAKKFVIYIKSVGIHYEVSEKYTTAPIQSTHTDFYSTCTGNCDTCRKNQALTGFQDFCVTPTSYWGCEEAWCFAINEGATCGFCRNIYDMDKSYRIYSVLKSTIVADVCISGILGGQCSRITEEVPYENTLFQADIQADLHNDGITIGELIAHGPDSHIYSGNIANLNDPVKMFGHPQLTHDGVPIFTKKTLEGDDMSWDCAAIGKKSVTIKTCGYDTYRFRSGLEQISDIPVSFKDFSSFFLAKSFSLGKLKMVVDLPSDLFKVAPKKPSITSTSLNCNGCLLCGQGLSCLLEFFSDLTFSTAISIDACSLSTYQLAVKKGSNKYNITMFCSANPDKKKMTLYPEGNPDISVEVLVNNVIVEEPENIIDQNDEYAHEEQQYNSDSSAWGFWDYIKSPFNFIASYFGSFFDTIRVVLLIAFIFLVTYFCSILTSICKGYVKNESYKSRSKIEDDDEPEIKAPMLMKDTMTRRRPPMDFSHLV

SEQ ID NO: 4 - protein, 285 aa, Tomato spotted wilt virus
AKVEIIRGDHPEIYDDSAENEVPTAASIQREAILETLTNLMLESRTPGTRQIREEKSTIPISAEPTTQKTISVLDLPNNCLNASSLKCEIKGISTYNVYYQVENNGVIYSCVSDSAEGLEKCDNSLNLPKRFSKVPVIPITKLDKKRHFSVGGKFFISESLTQDNYPITYNSYPTNGTVSLQTVKLSGDCKITKSNFANPYTVSITSPEKIMGYLIKKPGENVEHKVISFSGSASITFTEEMLDGEHNLLCGDKSAKIPKTNKRVRDCIIKYSKSIYKQTACINF

SEQ ID NO: 5 - DNA, 22 bp, ARTIFICIAL SEQUENCE
  PCR PRIMER SEQUENCE
ggtatcgtcctggactctggtg

SEQ ID NO: 6 - DNA, 19 bp, ARTIFICIAL SEQUENCE
  PCR PRIMER SEQUENCE
gggaagggcgtaaccttca

SEQ ID NO: 7 - DNA, 19 bp, ARTIFICIAL SEQUENCE
  PCR PRIMER SEQUENCE
gcttcccaccctttgattc

SEQ ID NO: 8 - DNA, 21 bp, ARTIFICIAL SEQUENCE
  PCR PRIMER SEQUENCE
atagccaagacaacactgatc